(19) United States
(12) Patent Application Publication
(10) Pub. No.: US 2025/0129404 A1
(43) Pub. Date: Apr. 24, 2025

(54) RAPID, COLORIMETRIC ASSAY TO DETECT ACETYLTRANSFERASE ACTIVITY IN BIOLOGICAL SAMPLES

(71) Applicant: THE FEINSTEIN INSTITUTES FOR MEDICAL RESEARCH, Manhasset, NY (US)

(72) Inventors: Sangeeta S. Chavan, Syosset, NY (US); Kevin J. Tracey, Old Greenwich, CT (US); Arielle H. Gabalski, Bellmore, NY (US)

(21) Appl. No.: 18/924,281

(22) Filed: Oct. 23, 2024

Related U.S. Application Data
(60) Provisional application No. 63/592,277, filed on Oct. 23, 2023.

(51) Int. Cl. C12Q 1/48

(52) U.S. Cl. CPC C12Q 1/48; G01N 2333/91057

(57) ABSTRACT

This disclosure relates to a rapid, colorimetric assay that detects choline acetyl transferase (hereafter “ChAT”) in biological samples. The inventive colorimetric assay can be used to measure and monitor extracellular levels of ChAT in a subject, such as a human or non-human animal, thereby improving the treatment of disease states where ChAT has been identified as a biomarker.

### CROSS REFERENCE TO RELATED APPLICATIONS

This application claims the benefit of and priority to U.S. Provisional Application No. 63/592,277, filed on Oct. 23, 2023, the entire contents of which being incorporated by reference herein in its entirety.

### INCORPORATION BY REFERENCE

All publications cited in this specification as well as the publications cited in said publications are hereby incorporated by reference. The discussion of these publications herein is intended merely to summarize the assertions made by applicant and no admission is made that any publication constitutes prior art.

### FIELD OF THE DISCLOSURE

This disclosure relates to a rapid, colorimetric assay that detects choline acetyl transferase (hereafter “ChAT”) in biological samples. The inventive colorimetric assay can be used to measure and monitor extracellular levels of ChAT in a subject, such as a human or non-human animal, thereby improving the treatment of disease states where ChAT has been identified as a biomarker. These disease states include: inflammatory diseases, such as, endotoxemia, inflammatory bowel disease, sepsis, and rheumatoid arthritis; disease states involving cognitive disorders, such as Alzheimer's disease (hereafter “AD”), Parkinson's disease, and ADHD; cancer; hypertension, and patients suffering from cardiac arrest. This disclosure further relates methods for improving the treatment of these disease states by using the inventive rapid, colorimetric assay to identify a subject suffering from a disease state where extracellular ChAT levels are implicated or to monitor the extracellular levels of ChAT in a subject during a course of treatment.

### BACKGROUND

Choline acetyl transferase (hereafter, “ChAT”) is the rate limiting enzyme in acetylcholine biosynthesis. Acetylcholine (hereafter “ACh”), the first neurotransmitter identified, plays an important role in a wide range of physiological processes, including muscle contraction, cardiovascular function, neural plasticity, attention, and memory. Although ACh is a well-known neurotransmitter, numerous studies have identified ChAT expression in a number of non-neuronal cells, including immune cells. ChAT-expression in immune cells has been linked to regulation of cytokine production, T cell responses, hematopoiesis, and blood pressure. ChAT-expressing T cells are required for antiviral immunity and vasomodulation during viral infection, whereas ChAT-expressing CD4+ T cells provide a cellular mechanism for suppression of TNF production during endotoxemia, and for vasorelaxation in the regulation of blood pressure. Further, acetylcholine produced by ChAT-expressing B lymphocytes regulates steady-state and emergency hematopoiesis. Moreover, in addition to ChAT-expressing CD4+ T cells, administration of recombinant ChAT to hypertensive animals normalizes blood pressure. Based on these observations it appears that circulating ChAT may be involved in regulation of inflammatory conditions.

ChAT catalyzes the biosynthesis of ACh in a highly efficient and rapid manner from choline and acetyl-CoA. As these substrates are adequately available in the cytoplasm, the level of ChAT is the rate-limiting step in ACh production. Although initially thought to be an exclusively intracellular enzyme, ChAT is released by a variety of cells including immune cells upon activation. Moreover, human embryonic cells and primary astrocytes in culture also release ChAT and high levels of functional ChAT have been observed within extracellular spaces such as cerebral spinal fluid, and plasma. ACh released by immune cells have been implicated in the regulation of inflammation. Although immune cells express a number of components of the cholinergic system, including ChAT and both muscarinic and nicotinic acetylcholine receptors, the expression of the vesicular ACh transporter (VAChT) is lacking, and it is not presently understood how acetylcholine is stored and released by these non-neuronal cells.

Extracellular ChAT activity has been linked to disease state involving inflammation. WO 2023/034110 A1 provides for the treatment of inflammatory diseases, such as endotoxemia, sepsis, and colitis, by administering an effective amount of a composition comprising ChAT or ChAT conjugated to polyethylene glycol (hereafter, “PEGylated ChAT”) and pharmaceutically acceptable carrier to a subject in need thereof. WO 2020/123611 provides for the administration of a composition comprising ChAT or PEGylated ChAT to treat hypertension. U.S. Pat. No. 11,298,408 B2 to Hanes et al., provides for the administration of CD4+ T-cell based compositions to treat hypertension in a subject. Gabalski et al. explores the administration of exogeneous ChAT an anti-inflammatory therapeutic agent (35).

Assays for measuring the activity of ChAT are known in the art. However, these assays are either immunological assays, which cannot measure the activity of ChAT, or employ reagents that prohibit its use on biological samples. S. Vijayaraghavan, et al. (PLOS One, vol. 8 (6), e65936 (June 2013)) describe an antibody-based assay for measuring ChAT and uses streptavidin-HRP. However, the inclusion of streptavidin-HRP may cause inaccurate reading because of non-specific interactions involving streptavidin-HRP with biotin in the biological samples. Kumar et al. (Scientific Reports, 6:931247 (August 2016)) and Steigler et al. (Mol. Med., vol. 27 (133) (2021)) describe a colorimetric assay for measuring the enzymatic activity ChAT. Kumar et al. uses phenol, 4-aminoantipyrine, and streptavidin-HRP to generate a chromophore that is monitored at 500 nm. Seigler et al. uses phenol, 4-aminoantipyrine, and horseradish peroxidase (hereafter, “HRP”), which also generates a chromophore 500 nm. Since, neither of these wavelengths can be used to assay biological samples (e.g., plasma) because of the color of plasma, especially at high concentrations, assay results are rendered unreliable. Fonnum (J. Neurochem., vol 19, pp. 407-409 (1975)) describes a radiochemical method to determine ChAT activity.

Hence, in view of the foregoing, there is a need for a rapid, colorimetric assay that can be used to determine ChAT activity in biological samples, which is simple to use, and which is much more sensitive than currently available options. Moreover, there is a need for a colorimetric assay in which the components are present in amounts that do not significantly alter enzymatic activity by changing, for example, the pH of the sample (e.g., physostigmine). These and other objectives are achieved by the present invention discussed below.

### SUMMARY

This present disclosure provides for a rapid, colorimetric assay to detect ChAT biomarker activity in biological samples taken from a subject (e.g., a human or a non-human animal). This disclosure further provides for a method for measuring and monitoring extracellular levels of ChAT in a human or non-human animal, thereby improving the diagnosis and/or treatment of disease states wherein ChAT has been identified as a biomarker.

Further, this disclosure provides for a method for screening or treating a disease in which an alteration in extracellular levels of ChAT, as compared to a subject or patient not suffering from the disease, is implicated, which comprises analyzing the extracellular levels of ChAT in a biological sample. Provided in accordance with the present disclosure are methods and compositions for detection of ChAT-biomarker associated diseases, using detection of biomarker ChAT activity as an indicator of the presence of said diseases.

Provided in accordance with the present disclosure are methods for detection of ChAT-biomarker associated diseases such as inflammatory disease, or cognitive disease, as well as detection and assessment of the severity of the disease comprising the measurement of ChAT activity. In addition, the disclosed methods and compositions may be used to determine whether a patient in treatment for ChAT-biomarker associated disease, such as an inflammatory disease, or cognitive disease, is responding to such treatments.

The present disclosure provides compositions and methods for laboratory and point-of-care tests for measuring ChAT activity in a sample from a subject. The compositions and methods can be generally applied for detection of a number of different diseases associated with an observed alteration in ChAT activity, e.g., ChAT-biomarker associated diseases. The ChAT assay method disclosed herein can be used in methods to diagnose, identify or screen subjects that may, or may not, be pre-disease state; to monitor subjects that are undergoing therapies for diseases associated with alterations in levels of ChAT activity; to determine or suggest a new therapy or a change in therapy; to evaluate the severity or changes in severity of the disease in a subject; to select or modify therapies or interventions for use in treating a subject with diseases associated with alterations in levels of ChAT. In an exemplary embodiment, the methods disclosed herein are used to identify and/or diagnose subjects who are asymptomatic or presymptomatic for a ChAT-biomarker associated disease such as an inflammatory, or cognitive disease.

In one embodiment a colorimetric assay for determining choline acetyl transferase (ChAT) activity in a biological sample is provided comprising (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form ACh in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity.

In another embodiment, a method for screening a patient for a ChAT mediated disease, choline acetyl transferase (ChAT) activity in a biological sample comprising: (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity; and (iii) determining whether the patient has a ChAT-biomarker associated disease based upon the observed ChAT activity.

In one aspect, a method is provided for treating a patient suffering from a ChAT-biomarker associated disease, comprising: (i) obtaining or having obtained a biological sample to be assayed for the presence of ChAT activity; (ii) performing or having performed a colorimetric assay for determining ChAT activity in the biological sample said assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm; and (iii) administering an effective amount of ChAT or an anticholinesterase agent to said patient based upon the observed ChAT activity.

In an embodiment, a method for screening a patient for a disease in which altered extracellular levels of ChAT are implicated, which comprises: (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form ACh in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity; and (iii) determining whether the patient has said disease in which altered extracellular levels of ChAT are implicated.

In an embodiment, a method is provided for treating a patient for a disease in which altered extracellular levels of ChAT are implicated which comprises which comprises: (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form ACh in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity; and (iii) administering an effective amount of ChAT or a therapeutic agent recognized to treat said disease to said patient based upon the choline acetyl transferase activity.

Another embodiment of the present invention provides for a prognostic method for determining whether a patient having a disease in which altered extracellular levels of ChAT are implicated, will respond to a drug treatment, which comprises: (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity; and (iii) assessing the prognosis in the patient based on the amount of observed ChAT activity in a sample before and after drug treatment. In an embodiment, increased levels of ChAT activity observed in the sample after drug treatment indicate a positive prognosis.

The disclosure further provides kits for performing any of the methods disclosed herein for a number of medical (including diagnostic and therapeutic) and research applications. In some embodiments, the kits are for determining therapy response in a subject. Kits may comprise a portable carrier, such as a box, carton, tube or the like, having in close confinement therein one or more containers, such as vials, tubes, ampoules, bottles, pouches, envelopes and the like. In various embodiments, a kit comprises one or more components selected from one or more media or media ingredients and reagents for the measurement of ChAT activity. For example, kits of the present disclosure may comprise, in the same or different containers, in any combination one or more suitable buffers and reagents any of which is described herein. The components may be contained within the same container or may be in separate containers to be admixed prior to use.

It is noted that in this disclosure and particularly in the claims and/or paragraphs, terms such as “comprises”, “comprised”, “comprising”, and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. Elements that are found to affect a basic or novel characteristic of the invention include the use of reagents or the use of reagents at concentrations that adversely affect measuring ChAT activity, such as by altering the pH (e.g., physostigmine) or cause non-specific interactions with proteins in the biological sample (e.g., streptavidin-HRP).

These and other embodiments are disclosed or are obvious from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1A-E: Active ChAT is present in normal serum and increases following an acute endotoxin challenge. (FIG. 1A) Concentration dependent detection of ChAT enzymatic activity in murine plasma samples obtained from normal mice. (FIG. 1B) Thermal denaturing of plasma at 95° C. for 30 minutes significantly attenuates ChAT activity (****P<0.0001). (FIG. 1C-E) Male C57BL/6 mice were given IP injection of lipopolysaccharide (LPS) or saline, and monitored and euthanized at various timepoints. Plasma TNF levels (FIG. 1C) were significantly elevated at 1.5 hours post LPS administration in comparison to saline (****P<0.0001). Sepsis score (FIG. 1D) was significantly elevated in mice that received LPS at all timepoints in comparison to saline administration. (FIG. 1E) ChAT activity (nmol/min/mg) increased significantly in mice given as compared to saline at 12 and 24 hours (*P<0.05) and 48 hours (****P<0.0001). Data were analyzed using a two-way ANOVA followed by Šídák's multiple comparisons test. (mean±SEM, n=5 to 10 per group).

FIG. 2: ChAT activity increases following cervical vagus nerve stimulation. Cervical Vagus Nerve stimulation (VNS) was performed on C57B16 mice for 4 mins (250 uA; 30 Hz; 250 ms pulse width). Mice recovered in home cage and 2 hours post stimulation mice were euthanized via exsanguination. ChAT activity increased significantly in VNS group compared to a sham control (*P<0.05). Data were analyzed using an unpaired t-test. (Data are represented as individual mouse data points with mean±SEM, n=5 to 10 per group).

FIG. 3A-D: ChAT administration attenuates inflammation, whereas Anti-ChAT administration exacerbates acute inflammation. C57/BL6 male mice were administered vehicle or 1 mg/kg PEG-ChAT or 1 mg/kg anti-ChAT antibody 30 minutes prior to 0.3 mg/kg lipopolysaccharide (LPS) injection. Ninety minutes later, mice were euthanized, and tissue was collected and assayed for TNF by ELISA. Administration of PEG-ChAT antibody significantly ameliorated serum TNF (FIG. 3A) compared to the vehicle group (**P<0.01). (FIG. 3B) Effect of PEG-ChAT on Spleen TNF. In contrast, administration of anti-ChAT antibody significantly increases serum (FIG. 3C) and spleen (FIG. 3D) TNF in comparison to vehicle treated (*P<0.05). Analyses are shown as mean±SEM. Data were analyzed using a Mann-Whitney t-test.

FIG. 4A-F: Administration of exogenous PEG-ChAT improved clinical disease outcomes in experimental colitis. (FIG. 4A) Experimental colitis was induced in C57Bl/6 male mice using 3% Dextran Sodium Sulfate solution for 7 days. Mice were monitored daily for 14 days and were administered by vehicle or PEG-ChAT intraperitoneally twice daily beginning on day 7. Administration of PEG-ChAT significantly reduces weight loss (FIG. 4B) and attenuates disease activity in comparison to vehicle treated animals (FIG. 4C). (FIG. 4D) PEG-ChAT improved the macroscopic appearance of harvested colons. (FIG. 4E) Effect of PEG-ChAT on macroscopic signs of colonic inflammation. Representative images of H&E stained colon swiss roll sections show that inflammation severity, crypt damage, and extent of inflammation is reduced in ChAT and PEG-ChAT treated mice. (FIG. 4F) Blinded histological scoring demonstrates reduced severity in the colons of mice receiving PEG-ChAT in comparison to vehicle. Data is expressed as mean±SEM. *P<0.05; **P<0.01.

FIG. 5A-C. Administration of PEG-ChAT attenuates serum pro-inflammatory cytokine levels in colitis. Blood was collected through cardiac puncture on day 14 of the model. Serum levels of IL-6 (FIG. 5A), TNF (FIG. 5B), and MCP-1 (FIG. 5C) levels were measured at day 14 of the model using MSD multiplex assay. Data is expressed as individual mouse data with mean±SEM. *P<0.05; **P<0.01.

FIG. 6. ChAT activity is increased in human serum during sepsis. Serum samples were obtained from (n=11) septic patients at the time of sepsis diagnosis, and (n=7) age-matched control patients. Serum ChAT activity (nmol/min/mg) is significantly increased in sepsis patients compared to controls. All data are represented as individual patient data points (a=patient who died from sepsis) with mean±SEM. ***P=0.0003.

FIG. 7A-F. Detection of recombinant ChAT activity. Representative images of the ChAT enzymatic assay, choline standard and the measured activity in a dose response using in-house prepared recombinant ChAT protein (rChAT) (FIG. 7A). A significant increase in enzymatic activity of the rChAT is observed compared to a commercially available ChAT (FIG. 7B) (**P<0.01). The Michaelis-Menten approximation (FIG. 7C) and Lineweaver Burke (FIG. 7D) double-reciprocal relationships of rChAT (2 μg/mL) show that the Vmax=0.05 Δ CoA (nmol/min) and the Km=247.4 (nmol) for choline respectively. Preincubation of 0.4 mg/mL rChAT with a competitive inhibitor alpha-NETA demonstrated an IC₅₀ of 0.1485 μM (FIG. 7E). Thermal denaturing of rChAT at 70° C. for 30 minutes significantly reduced the detected activity in comparison to native preparation (FIG. 7F) (**P<0.01).

FIG. 8. The levels of extracellular ChAT increase in patients suffering from sepsis.

FIG. 9. The levels of extracellular ChAT increase in patients suffering from ADHA.

### DETAILED DESCRIPTION

This present disclosure provides for a rapid, colorimetric assay to detect ChAT biomarker activity in biological samples taken from a subject (e.g., a human or a non-human animal). As disclosed herein, altered levels of ChAT biomarker activity are associated with a number of diseases, herein referred to as ChAT-biomarker associated diseases. Non-limiting ChAT-biomarker associated diseases, in which altered extracellular levels of ChAT are implicated, include inflammatory diseases, such as, endotoxemia, inflammatory bowel disease, sepsis, and rheumatoid arthritis; and disease states involving cognitive disorders, such as AD, Parkinson's disease, and ADHD. The provided assay methods may also be used to detect cancer; hypertension; and cardiac arrest in subjects. In specific embodiments, the ChAT-biomarker associated diseases include Alzheimer's disease, ADHD and sepsis.

Provided in accordance with the present disclosure are methods and compositions for detection of ChAT-biomarker associated diseases, using detection of biomarker ChAT activity as an indicator of the presence of said diseases. Provided in accordance with the present disclosure are methods for detection and assessment of the severity of the ChAT-biomarker associated diseases and whether a patient in treatment for said disease, is responding to such treatments. In an embodiment said ChAT-biomarker associated diseases are inflammatory or cognitive diseases.

The present disclosure provides compositions and methods for laboratory and point-of-care tests for measuring ChAT activity in a sample from a subject. The compositions and methods can be generally applied for detection of a number of different diseases associated with an observed alteration in ChAT activity, e.g., ChAT-biomarker associated diseases. The ChAT assay method disclosed herein can be used in methods to diagnose, identify or screen subjects that may, or may not, be pre-disease state; to monitor subjects that are undergoing therapies for diseases associated with altered levels of ChAT activity; to determine or suggest a new therapy or a change in therapy; to evaluate the severity or changes in severity of the disease in a subject; to select or modify therapies or interventions for use in treating a subject with diseases associated with altered levels of ChAT. In an exemplary embodiment, the methods disclosed herein are used to identify and/or diagnose subjects who are asymptomatic or presymptomatic for a ChAT-biomarker associated disease such as an inflammatory, or cognitive disease.

The term “biomarker” refers to a molecule whose measurement provides information as to the state of a subject. In various exemplary embodiments, the ChAT biomarker is used to assess a pathological state, e.g., ChAT-biomarker associated diseases such as inflammation and/or cognitive disorders. Measurements of the biomarker may be used alone or combined with other data obtained regarding a subject in order to determine the pathological state of the subject. In one embodiment, the biomarker is “differentially present” in a sample taken from a subject of one phenotypic status (e.g., having a ChAT-biomarker associated disease associated with altered levels of ChAT activity) as compared with another phenotypic status (e.g., not having said disease). The biomarker may be “differentially present” even if there is no phenotypic difference, e.g. the biomarker may allow the detection of asymptomatic risk. A biomarker may be determined to be “differentially present” in a variety of ways, for example, between different phenotypic statuses if the mean or median level or concentration (particularly the expression level of ChAT activity as described below) of the biomarker in the different groups is calculated to be statistically significant. Common tests for statistical significance include, among others, t-test, ANOVA, Kruskal-Wallis, Wilcoxon, Mann-Whitney and odds ratio.

The compositions and methods of the present disclosure can be used in the prognosis, diagnosis and treatment of ChAT-biomarker associated diseases such as, for example, inflammation or cognitive diseases, in a subject. A “subject” in the context of the present disclosure is an animal, preferably a mammal. The mammal can be a human, non-human primate, mouse, rat, dog, cat, horse, or cow, but are not limited to these examples. In various exemplary embodiments, a subject is human and may be referred to as a “patient”. Mammals other than humans can be advantageously used as subjects that represent animal models of ChAT-biomarker associated diseases or for veterinarian applications. A subject can be one who has been previously diagnosed or identified as having a ChAT-biomarker associated diseases, and optionally has already undergone, or is undergoing, a therapeutic intervention for said disease. Alternatively, a subject can also be one who has not been previously diagnosed as having said diseases. For example, a subject can be one who exhibits one or more risk factors for diseases, or one who does not exhibit a disease risk factor, or one who is asymptomatic for said disease. In certain embodiments, the subject can be already undergoing therapy or can be a candidate for therapy.

The term “sample” used herein refers to a specimen or culture obtained from a subject and includes a fluid obtained from a subject including, for example, whole blood or a blood derivative (e.g. serum, plasma, or blood cells), ovarian cyst fluid, ascites, lymphatic, cerebrospinal or interstitial fluid, saliva, mucous, sputum, sweat, urine, or any other secretion, excretion, or other bodily fluids. As will be appreciated by those in the art, virtually any experimental manipulation or sample preparation steps may have been done on the sample. Methods for collection of biological samples for use in diagnostic methods are well known to those of skill in the art.

The inventive colorimetric assay provided herein involves three successive chemical reactions:

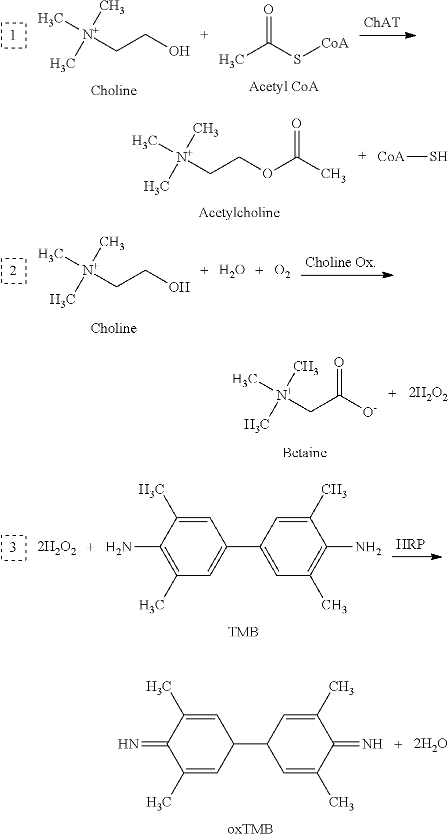

In the first of these reactions, ChAT catalyzes the acetylation of the limiting substrate, choline, to form ACh. In the second reaction, choline oxidase (“Choline Ox”) oxidizes the remaining choline to produce hydrogen peroxide. In the third reaction, horseradish peroxidase (“HRP”) oxidizes the substrate 3,3′,5,5′-tetramethylbenzidine (“TMB”) relative to the quantity of hydrogen peroxide produced in reaction; this results in a colored product that can be assessed at 650 nm.

Accordingly, the present disclosure provides a colorimetric assay for determining choline acetyl transferase (ChAT) activity in a biological sample comprising: comprising (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity. In a non-limiting embodiment, the assay is conducted in a multi-well plate. In another non-limiting embodiment, the subject sample may be subjected to serial dilutions.

In one embodiment, the present disclosure provides for a colorimetric assay for determining choline acetyl transferase (ChAT) activity in a biological sample. The inventive colorimetric assay is illustrated by the following non-limiting procedure. Biological samples and choline chloride standards are diluted in Assay Buffer. 100 μl of each standard and 40 μl of each sample are added in triplicate to a reaction plate, such as, for example 96-well plate. Reaction plates containing different numbers of wells may be used depending upon the circumstances. In the wells containing 40 μl of samples, 60 μl of Cocktail A are added. After incubation at 37° C. for 20 minutes, 50 μl of Cocktail B are added to all wells and the plate is incubated at room temperature for 15 minutes. The assay is developed by addition of 150 μL of TMB to all wells, and the plate is immediately analyzed spectrophotometrically at 650 nm.

In the above assay, and any additional assays disclosed herein that refer to an Assay Buffer, Cocktail A and Cocktail B said reagents are as follows: the Assay Buffer is a solution of 10 mM Tris-HCl, 150 mM NaCl, 1 mM EDTA, and 0.05% Triton A. Cocktail A is a solution of 250 μM choline chloride, 500 M acetyl coenzyme A, and 20 μM physostigmine in Assay Buffer. Cocktail B is a solution of 1 U/ml choline oxidase and 50 U/ml of horseradish peroxidase in Assay Buffer. The Reaction Standard is a solution that contains 0 nmol to 50 nmol choline in Assay Buffer.

In an embodiment, a method is provided for screening a patient for a ChAT-biomarker associated disease, comprising: (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity; and (iii) determining whether the patient has a ChAT-biomarker associated disease based upon the observed ChAT activity. In an embodiment, an alteredlevel of ChAT activity indicates that the patient has a ChAT biomarker associated disease.

In a non-limiting embodiment the present disclosure provides for a method for screening a patient for a ChAT-biomarker associated disease, which comprises: (i) obtaining or having obtained a biological sample from the patient; and (ii) performing or having performed a colorimetric assay for determining choline acetyl transferase activity on the biological sample comprising: (a) preparing at least one test sample by taking the biological sample and diluting said biological sample with Assay Buffer; (b) adding 40 μL of the at least one test sample to a reaction well in a multi-well reaction plate, which further comprises reaction wells that comprise 100 μL of at least one Reaction Standard; (c) adding 60 μL of Cocktail A to the at least one plasma sample; (d) incubating the reaction plate at about 35-40° C. for about 15-20 minutes; (e) adding 50 μL of Cocktail B to all reaction wells; (f) incubating the reaction plate at room temperature for about 15-20 minutes; (g) adding 150 μL of 3,3′,5,5′-tetramethylbenzidine to all reaction wells; and (h) analyzing the reaction plate spectrophotometrically at 650 nm; and
(iii) determining whether the patient has a ChAT-biomarker associated disease based upon the observed ChAT activity. In an embodiment, an altered level of ChAT activity indicates that the patient has a ChAT biomarker associated disease.

Provided is a method for screening a patient for a disease in which altered extracellular levels of ChAT are implicated, which comprises: (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form ACh in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity; and (iii) determining whether the patient has said disease in which altered extracellular levels of ChAT are implicated.

In a specific embodiment, a method for screening a patient for a disease in which altered extracellular levels of ChAT are implicated, which comprises: (a) obtaining or having obtained a biological sample from the patient; and (b) performing or having performed a colorimetric assay for determining choline acetyl transferase activity on the biological sample comprising: (i) preparing at least one test sample by taking the biological sample and diluting said biological sample with Assay Buffer; (ii) adding 40 μL of the at least one test sample to a reaction well in a multi-well reaction plate, which further comprises reaction wells that comprise 100 μL of at least one Reaction Standard; (iii) adding 60 μL of Cocktail A to the at least one plasma sample; (iv) incubating the reaction plate at about 35-40° C. for about 15-20 minutes; (v) adding 50 μL of Cocktail B to all reaction wells; (vi) incubating the reaction plate at room temperature for about 15-20 minutes; (vii) adding 150 μL of 3,3′,5,5′-tetramethylbenzidine to all reaction wells; and (viii) analyzing the reaction plate spectrophotometrically at 650 nm; and (c) determining whether the patient has said disease based upon the choline acetyl transferase activity.

In various exemplary embodiments, detection and measurement of ChAT activity utilizes colorimetric methods and systems in order to provide an indication of extracellular enzyme activity. In colorimetric methods, the presence of ChAT activity will result in a change in the absorbance or transmission of light by a sample at one or more wavelengths. Detection of the absorbance or transmission of light at such wavelengths thus provides an indication of the presence of the target species.

A detection system for colorimetric methods includes any device that can be used to measure colorimetric properties. Generally, the device is a spectrophotometer, a colorimeter or any device that measures absorbance or transmission of light at one or more wavelengths. In various embodiments, the detection system comprises a light source; a wavelength filter or monochromator; a sample container such as a cuvette or a reaction vial; a detector, such as a photoresistor, that registers transmitted light; and a display or imaging element. In some embodiments, a change in the colorimetric properties of a sample can be detected directly by the naked eye, i.e., by direct visual inspection. In a non-limiting embodiment, the assay samples are analyzed by using a spectrophotometer at 650 nm.

The inventive colorimetric assay has many advantages over assays known in the prior art. First, the inventive chromogen substrate is spectrophotometrically not read at a wavelength in which the color of plasma will interfere. This stands in contrast to prior assays, which had the potential of providing unreliable results, especially at high concentrations of plasma. Moreover, the inventive assay does not require the samples to be denatured and used as a control. This reduces the assay procedure time, reagent requirements, and the amount of the biological sample required. Further, by avoiding streptavidin-HRP, nonspecific reactions between streptavidin and biotin are avoided. Additionally, the inventive method avoids obtaining false-positive readings due to hemoglobin color. Moreover, the reagents are present in amounts that permit a more sensitive detection compared to other methods since the amounts used do not affect the activity of ChAT.

In an embodiment, for the diagnostic methods disclosed herein, an alteration in observed ChAT activity, indicates that the patient has a ChAT-biomarker associated disease. In a non-limiting embodiment, the ChAT-biomarker associated disease may be a cognitive disorder such as that associated with Huntington disease, stroke, multiple sclerosis, Parkinson disease, Lewy body dementia, meningitis, acquired immune deficiency syndrome, alcohol, drugs, and toxins. In a specific embodiment, the cognitive disorder is Alzheimer's disease. In another embodiment, the ChAT-biomarker associated disease may be an inflammatory disease such as endotoxemia, inflammatory bowel disease, sepsis, and rheumatoid arthritis. In a specific embodiment, the inflammatory disease is sepsis.

ChAT activity is determined from the interpolated amount of choline depleted during the reaction, divided by the reaction time and sample volume, and multiplied by the sample dilution factor according to the following equation:

$$Choline Depleted ( \frac{\frac{nmol}{min}}{mL} ) = \frac{[ ( [ Choline Blank ] ) - ( [ P lasma Sample ] ) ]}{Reaction Time ( mins ) * S ample Volume ( mL )} \times DF$$

where DF is the dilution factor. ChAT activity is normalized to total protein concentration, measured using Bradford protein assay (Bio-Rad) and is represented as nmol/min/mg of total protein.

The present disclosure provides an innovative assay method for detection and measurement of ChAT activity. The term “measuring,” “detecting,” or “taking a measurement” refers to a quantitative or qualitative determination of a property or characteristic of an entity, e.g., quantifying the amount or the activity level of a molecule. The term “concentration” or “level” can refer to an absolute or relative quantity. Measuring a molecule may also include determining the absence or presence of the molecule.

As disclosed herein, alterations in levels of ChAT activity can be associated with the presence of ChAT-biomarker associated diseases. As disclosed herein, altered levels of ChAT biomarker activity are associated with a number of diseases when compared to a subject or patient not suffering the disease (herein referred to as “ChAT-biomarker associated disease”); for example, the amount of ChAT may be below a standard, reference range or above a standard, reference range, depending upon the specific ChAT-biomarker associated disease. For example, for AD, the standard, reference level of ChAT activity could be less than about 2 nmol/min/mg. In another aspect, for sepsis, the standard, reference level of ChAT activity could be above about 5 nmol/min/mg. In another aspect, for ADHD, the standard, reference level of ChAT activity could be above about 0.5 mmol/min/mg

In some embodiments, the sample (e.g. blood, serum or plasma) levels of biomarker ChAT activity will be altered in a subject as compared to a normal subject indicating presence, or predisposition, to a ChAT-biomarker associated disease. In a non-limiting embodiment, the disease is an inflammatory or cognitive disease. In some embodiments, these changes will be about 1% to about 90%, 10% to about 80%, 20% to about 70%, about 30% to about 60% or about 40% to about 50% from a reference value. In some embodiments, these changes will be about 10% to about 20%, about 10% to about 30%, about 10% to about 40%, about 10% to about 50%, about 10% to about 60%, about 10% to about 70%, or about 10% to about 90% from a reference value. In some embodiments, changes of at least about a percentage selected from 1%, 5%, 10%, 11%, 12%, 13%, 14%, 15%, 16%, 17%, 18%, 19%, 20%, 30%, 40%, 50%, 60%, 70%, 80%, 90%, 95%, 96%, 97%, 98% and 99% from a reference value will occur.

Subjects identified as having a disease or being at increased risk of developing a disease based on detection of alterations in ChAT activity may be chosen to receive a therapeutic regimen to slow the progression of a disease or decrease or prevent the risk of developing a disease. The levels of the ChAT biomarker may be used by physicians to design patient-specific treatment protocols that are most likely to result in disease treatments, including, for example, treatment of ChAT-biomarker associated diseases. Specifically, the methods comprise correlating diagnostic or prognostic results, e.g., detection of biomarker presence and quantity, with disease severity or to other clinical parameters such as predicted response to treatment and overall survival. In a specific embodiment, the methods comprise correlating the diagnostic results to the therapeutic outcome of ChAT-biomarker associated disease treatment.

The novel colorimetric assay disclosed herein can be used by a practitioner to determine and effect appropriate therapies with respect to a subject given the disease status indicated by measurements of the ChAT biomarker in a sample from the subject. Thus, in one aspect, the disclosure provides a method of treating a disease in a subject comprising taking a measurement of the ChAT biomarker in a sample from the subject and effecting a therapy with respect to the subject. In one embodiment, the concentration of the biomarker increases or decreases according to the values described herein or stay the same in response to the therapy.

In an embodiment, the present disclosure provides methods for treatment of ChAT-biomarker associated disease comprising administration of an effective amount of a therapeutic agent to relieve the symptoms of the disease. The term “ChAT-biomarker associated disease” as used herein means a condition or disease characterized by altered levels of ChAT activity as compared to a subject or patient not suffering from the disease. The identification and selection of a specific therapeutic agent to treat a specific disease in which altered extracellular levels of ChAT are implicated would be well within the skill level of a person have ordinary skill in the art.

In some embodiments the disease may result from under expression of the ChAT protein. In disorders characterized by low levels of the ChAT protein, the ChAT protein may be administered as a therapeutic agent to increase the levels of circulating the ChAT protein. Sources of ChAT include commercially available ChAT, or recombinantly expressed ChAT. In an embodiment the ChAT is pegylated ChAT.

When the therapeutic agent is ChAT, the therapeutic agent may be the ChAT protein itself or a modified ChAT protein such as a PEGylated ChAT. The terms “protein” used herein, unless specified to the contrary, and according to conventional meaning, means a sequence of amino acids. Proteins are not limited to a specific length, e.g., they may comprise a full-length protein sequence or a fragment of a full-length protein, and may include post-translational modifications of the polypeptide, for example, glycosylations, acetylations, phosphorylations and the like, as well as other modifications known in the art, both naturally occurring and non-naturally occurring, e.g. variants. In another embodiment, the therapeutic agent is one that results in increased levels of ChAT enzyme activity. In another embodiment, the therapeutic agent is an anticholinesterase agent that may be used in the inventive methods, and which are well known in the art. Non-limiting examples include donepezil, galantamine, pyridostigmine and rivastigmine.

In some embodiments the disease may result from over expression of the ChAT protein. In disorders characterized by increased levels of the ChAT protein, antagonists of the ChAT protein may be administered to decrease the levels of circulating the ChAT activity. Such antagonists include, for example, anti-ChAT antibodies or anti-sense ChAT molecules. The identification and selection of a specific therapeutic agent to treat a specific disease in which increased extracellular levels of ChAT are implicated would be well within the skill level of a person having ordinary skill in the art.

The term “therapeutic agent” as used herein is a compound capable of producing a desired and beneficial effect. The terms “treat,” “treating” or “treatment” of any disease or disorder as used herein refer in one embodiment, to halting the progression of the condition or disease, or to ameliorating the disease or disorder (i.e., slowing or arresting or reducing the development of the disease or at least one of the clinical symptoms thereof). In another embodiment “treat,” “treating” or “treatment” refers to alleviating or ameliorating at least one physical parameter including those which may not be discernible by the patient. In yet another embodiment, “treat,” “treating” or “treatment” refers to modulating the disease or disorder, either physically, (e.g., stabilization of a discernible symptom), physiologically, (e.g., stabilization of a physical parameter), or both. In yet another embodiment, “treat,” “treating” or “treatment” refers to preventing or delaying the onset or development or progression of the disease or disorder. As used herein, a subject is “in need of” a treatment if such subject would benefit biologically, medically or in quality of life from such treatment.

Pharmaceutical compositions of embodiments comprise a therapeutically effective amount of a therapeutic agent, such as for example, ChAT protein, anticholinesterase agents, or an antagonist of ChAT activity, dissolved or dispersed in a pharmaceutically acceptable carrier. The preparation of a pharmaceutical composition that contains the ChAT protein and optionally an additional active ingredient will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred compositions are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The terms “effective amount” or “therapeutically effective amount” as used herein have the standard meanings known in the art and are used interchangeably herein to mean an amount sufficient to treat a subject afflicted with a condition or disease (e.g., ChAT-biomarker associated diseases) or to halt the progression of the condition or disease, or alleviate a symptom or a complication associated with the condition or disease. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). For example, in the case of an agent treating a ChAT-biomarker associated disease, an effective amount may be an amount sufficient to result in clinical improvement of the patient.

The terms “therapy” and “treatment” may be used interchangeably. In certain embodiments, the therapy can be selected from, without limitation, initiating therapy, continuing therapy, modifying therapy or ending therapy. A therapy also includes any prophylactic measures that may be taken to prevent ChAT-biomarker associated diseases such as inflammation or cognitive disorders. In exemplary embodiments, effecting a therapy comprises administering a ChAT-biomarker associated disease modulating drug to a subject.

Accordingly, in an embodiment a method is provided for treating a patient suffering from a ChAT-biomarker associated disease, comprising: (i) obtaining or having obtained a biological sample to be assayed for the presence of ChAT activity; (ii) performing or having performed a colorimetric assay for determining ChAT activity in the biological sample said assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm; and (iii) administering an effective amount of a therapeutic agent to said patient based upon the observed ChAT activity. In an embodiment, the therapeutic agent is, for example, ChAT, an anticholinesterase agent or a ChAT antagonist.

More specifically, a method is provided for treating a patient suffering from a ChAT-biomarker associated disease which comprises: (a) obtaining or having obtained a biological sample from the patient; and (b) performing or having performed a colorimetric assay for determining choline acetyl transferase activity in the biological sample comprising: (i) preparing at least one test sample by taking the biological sample and diluting said biological sample with Assay Buffer; (ii) adding 40 μL of the at least one test sample to a reaction well in a multi-well reaction plate, which further comprises reaction wells that comprise 100 μL of at least one Reaction Standard; (iii) adding 60 μL of Cocktail A to the at least one plasma sample; (iv) incubating the reaction plate at about 35-40° C. for about 15-20 minutes; (v) adding 50 μL of Cocktail B to all reaction wells; (vi) incubating the reaction plate at room temperature for about 15-20 minutes; (adding 150 μL of 3,3′,5,5′-tetramethylbenzidine to all reaction wells; (vii) analyzing the reaction plate spectrophotometrically at 650 nm; and (c) administering an effective amount of a therapeutic agent to said patient based upon the observed ChAT activity. In an embodiment, for example, the therapeutic agent is ChAT, an anticholinesterase agent or a ChAT antagonist.

Further, a method is provided for treating of a patient for a disease in which altered extracellular levels of ChAT are implicated which comprises which comprises: (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity; and (iii) administering an effective amount of therapeutic agent based upon the choline acetyl transferase activity. In an embodiment, the therapeutic agent is ChAT, an anticholinesterase agent, a ChAT antagonist or a therapeutic agent recognized to treat said disease to said patient.

In an embodiment, a method is provided for treating of a patient for a disease in which altered extracellular levels of ChAT are implicated which comprises: (a) obtaining or having obtained a biological sample from the patient; and (b) performing or having performed a colorimetric assay for determining choline acetyl transferase activity in the biological sample comprising: (i) preparing at least one test sample by taking the biological sample and diluting said biological sample with Assay Buffer; (ii) adding 40 μL of the at least one test sample to a reaction well in a multi-well reaction plate, which further comprises reaction wells that comprise 100 μL of at least one Reaction Standard; (iii) adding 60 μL of Cocktail A to the at least one plasma sample; (iv) incubating the reaction plate at about 35-40° C. for about 15-20 minutes; (v) adding 50 μL of Cocktail B to all reaction wells; (vi) incubating the reaction plate at room temperature for about 15-20 minutes; (vii) adding 150 μL of 3,3′,5,5′-tetramethylbenzidine to all reaction wells; and (viii) analyzing the reaction plate spectrophotometrically at 650 nm; and (c) administering an effective amount of therapeutic agent based upon the choline acetyl transferase activity. In embodiment, the therapeutic agent is ChAT, an anticholinesterase agent, a ChAT antagonist or a therapeutic agent recognized to treat said disease to said patient.

Measurement of biomarker concentrations also allows for the course of treatment to be monitored. The effectiveness of a treatment regimen can be monitored by detecting ChAT activity from samples obtained from a subject over time and comparing the amount of biomarker detected. For example, a first sample can be obtained prior to the subject receiving treatment and one or more subsequent samples are taken after or during treatment of the subject. Changes in biomarker concentration across the samples may provide an indication as to the effectiveness of the therapy. To identify therapeutics or drugs that are appropriate for a specific subject, biomarker concentrations can be compared to a sample derived from the subject before and after treatment or exposure to a therapeutic agent or a drug or can be compared to samples derived from one or more subjects who have shown improvements relative to a disease as a result of such treatment or exposure.

In an embodiment, a method is provided for identifying an effective drug treatment for a patient having a disease in in which altered extracellular levels of ChAT are implicated, comprising: (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity; and (iii) identifying whether a drug treatment is effective based on the amount of observed ChAT activity. In an embodiment, an alteration in ChAT activity within a sample is indicative of an effective drug treatment.

Another embodiment of the present invention provides for a method for identifying an effective drug treatment for a patient having a disease in which altered extracellular levels of ChAT are implicated, which comprises: (i) obtaining or having obtained a biological sample from the patient prior to the patient receiving the drug treatment and one or more samples after or during drug treatment; and (ii) performing or having performed a colorimetric assay for determining observed ChAT activity in the biological samples comprising: (a) preparing at least one test sample by taking the biological sample and diluting said biological sample with Assay Buffer; (b) adding 40 μL of the at least one test sample to a reaction well in a multi-well reaction plate, which further comprises reaction wells that comprise 100 μL of at least one Reaction Standard; (c) adding 60 μL of Cocktail A to the at least one plasma sample; (d) incubating the reaction plate at about 35-40° C. for about 15-20 minutes; (e) adding 50 μL of Cocktail B to all reaction wells; (f) incubating the reaction plate at room temperature for about 15-20 minutes; (g) adding 150 μL of 3,3′,5,5′-tetramethylbenzidine to all reaction wells; and (h) analyzing the reaction plate spectrophotometrically at 650 nm; and (iii) identifying whether a drug treatment is effective based on the amount of observed ChAT activity. In an embodiment, an alteration in ChAT activity within a sample is indicative of an effective drug treatment.

In an embodiment, a prognostic method for determining whether a patient having a disease in which altered extracellular levels of ChAT are implicated, will respond to a drug treatment, comprising: (i) obtaining or having obtained a biological sample to be assayed for the presence of ChAT activity; (ii) performing a colorimetric assay for determining ChAT activity in the biological sample said colorimetric assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity; and (iii) identifying whether the patient will respond to a drug treatment.

Another embodiment of the present invention provides for a prognostic method for determining whether a patient having a disease in which altered extracellular levels of ChAT are implicated, will respond to a drug treatment, which comprises: (i) obtaining or having obtained a biological sample from the patient prior to the patient receiving the drug treatment and one or more samples after or during drug treatment; and (ii) performing or having performed a colorimetric assay for determining observed ChAT activity in the biological samples comprising: (a) preparing at least one test sample by taking the biological sample and diluting said biological sample with Assay Buffer; (b) adding 40 μL of the at least one test sample to a reaction well in a multi-well reaction plate, which further comprises reaction wells that comprise 100 μL of at least one Reaction Standard; (c) adding 60 μL of Cocktail A to the at least one plasma sample; (d) incubating the reaction plate at about 35-40° C. for about 15-20 minutes; (e) adding 50 μL of Cocktail B to all reaction wells; (f) incubating the reaction plate at room temperature for about 15-20 minutes; (g) adding 150 μL of 3,3′,5,5′-tetramethylbenzidine to all reaction wells; and (h) analyzing the reaction plate spectrophotometrically at 650 nm; and
(iii) assessing the prognosis in the patient based on the amount of observed ChAT activity in a sample before and after drug treatment. In an embodiment, altered levels of ChAT activity observed in the sample after drug treatment indicates a positive prognosis.

The disclosure further provides kits for performing any of the methods disclosed herein for a number of medical (including diagnostic and therapeutic) and research applications. In some embodiments, the kits are for determining therapy response in a subject. Kits may comprise a portable carrier, such as a box, carton, tube or the like, having in close confinement therein one or more containers, such as vials, tubes, ampoules, bottles, pouches, envelopes and the like. In various embodiments, a kit comprises one or more components selected from one or more media or media ingredients and reagents for the measurement of ChAT activity. For example, kits of the present disclosure may comprise, in the same or different containers, in any combination one or more suitable buffers and reagents any of which is described herein. The components may be contained within the same container or may be in separate containers to be admixed prior to use.

The kits of the present disclosure may also comprise one or more instructions or protocols for carrying out the methods of the present disclosure. Generally, any of the methods disclosed herein can comprise using any of the kits disclosed herein. In a non-limiting embodiment, the kit for determining the cholinesterase activity in a biological sample, which comprises Cocktail A, Cocktail B, and Assay Buffer.

### Example

Certain embodiments of the invention will now be illustrated by the following non-limiting example.

Physiological anti-inflammatory mechanisms represent efficient processes which are conserved through evolution, to regulate inflammatory conditions. Understanding these mechanisms provides novel strategies which can be exploited for the treatment of inflammatory disorders. As disclosed herein circulating ChAT is a regulator of inflammation. ChAT activity increases in the setting of acute inflammation in proportion to illness severity. This increase provides a therapeutic physiological response, as administration of a neutralizing antibody for ChAT amplifies the inflammatory response to endotoxin. Conversely, increasing ChAT activity by systemic administration of ChAT suppresses inflammation both acutely, as well as in a model of inflammatory bowel disease. Vagus nerve stimulation, which is well known to activate cholinergic anti-inflammatory responses, induces an increase in plasma ChAT activity. Finally, the observation that ChAT serum activity appears to be increased in patients with sepsis indicates that increased circulating ChAT may be a response to cytokine storm.

In addition to the data described below, significant increases in ChAT activity have been observed in the cerebral spinal fluid (CSF) of patients diagnosed with multiple sclerosis and have found changes in both the CSF and plasma of patients with dementias of different etiologies, including Alzheimer's disease (13, 27). The source of this extracellular ChAT is currently unclear. Numerous studies have demonstrated the presence of ChAT in human and rodent lymphoid cells (28-30). Stimulated splenic lymphocytes release ChAT, so potentially circulating lymphocytes which are subject to inflammatory stimulation may release ChAT into the circulation (13). B cells have also recently been documented to synthesize acetylcholine for use in controlling hematopoiesis in the bone marrow which is a compartment of the systemic circulation (9, 31). In addition to lymphocytes, monocytes also express ChAT which is upregulated in renal allograft rejection, and a small fraction of NK cells at baseline express ChAT which is upregulated by inflammation and has been documented to modulate the activity of monocytes and macrophages in brain inflammation (32).

Therefore, given the plethora of immune cells expressing ChAT, it is likely that circulating leukocytes are a source of plasma ChAT activity. Alternatively, or in addition to these components of the immune system, endothelial cells also express ChAT which given their continuous exposure to the blood may release ChAT when subjected to inflammatory stressors (33). Finally, mesenchymal cells have been documented to secrete acetylcholine subsequently modulating inflammation (34). Given the widespread non-neuronal expression of ChAT, it is likely that other non-neuronal cell types may also express ChAT which could be secreted to participate in protective anti-inflammatory responses. The disclosure herein of circulating extracellular ChAT contributing to regulation of inflammation reveals a previously unknown mechanism in immune homeostasis. The observation that neutralization of circulating ChAT results in an increased inflammatory phenotype in mice indicates that circulating ChAT plays a homeostatic role in regulation of inflammation. From this perspective, circulating ChAT may make acetylcholine available to cells devoid of direct cholinergic innervation using choline and acetyl-CoA within the plasma to produce acetylcholine directly at the target site, providing a biochemical mechanism for acetylcholine-mediated immune regulation.

In sum, these observations establish a role for circulating ChAT in the regulation of inflammatory processes and indicate that enhance acetylcholine release in the circulation may be enhanced to therapeutically regulate inflammation, insights that can have implications for developing a novel therapy for inflammatory diseases. Moreover, the data presented below that circulating ChAT activity is regulated by signals transmitted in the vagus nerve can offer another strategy to control the activity of ChAT using neuromodulation strategies and prevent or regulate inflammation.

### Material and Methods

Recombinant ChAT protein and ChAT PEGylation. All recombinant ChAT (rChAT) protein used was produced as described previously (10). Briefly, recombinant human ChAT corresponding to residue 119-748 (EC2.3.1.6, UnitProt 28329-3) with a N-histidine tag was expressed in E. coli BL21-Gold (DE3) cells and purified using high affinity Ni-charged columns. The purified recombinant ChAT was dialyzed at 4° C. in buffer containing 0.2×PBS, 10% glycerol and 0.5 mM TCEP (tris(2-carboxyethyl) phosphine), and subjected to extensive Triton X-114 extraction to remove contaminating endotoxins. After purification, PEGylation was carried out using MS (PEG) 12 reagent to extend the plasma half-life of the enzyme (10). Following PEGylation, the protein was dialyzed in buffer and once again extracted with Triton-X-114 to remove contaminating endotoxins. A Bradford protein assay (Bio-Rad) was used to determine rChAT protein concentration. Commercial recombinant ChAT protein was purchased from My BioSource.

Choline Acetyltransferase activity assay. An assay was developed to measure ChAT activity using an adapted version of the colorimetric assay (Reactions 1-3) (13).

The inventive colorimetric assay involves three successive chemical reactions:

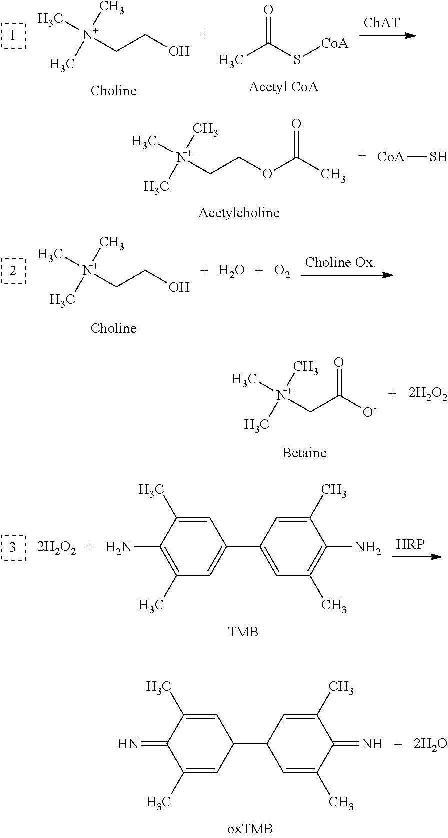

Briefly, biological samples and choline chloride standards are diluted in assay buffer (containing 10 mM Tris-HCl, 150 mM NaCl, 1 mM EDTA, and 0.05% Triton X-100). 100 μl of each standard and 40 μl of each sample are added in triplicate to a 96-well plate. In wells containing 40 μl of samples, 60 μl of Cocktail A (250 μM Choline chloride, 500 μM acetyl coenzyme A, and 10 μM physostigmine in assay buffer) are added. After incubation at 37° C. for 20 minutes, 50 μl of Cocktail B (1 U/ml choline oxidase, 50 U/ml HRP in Assay Buffer) are added to all wells and the plate is incubated at room temperature for 15 minutes. The assay is developed by addition of 150 μL of 3′3′5′5′ Tetramethylbenzidine (TMB) to all wells, and the plate is immediately analyzed spectrophotometrically at 650 nm. ChAT Activity is determined from the interpolated amount of choline depleted during the reaction, divided by the reaction time and sample volume, and multiplied by the sample dilution factor (Equation 1). In all biological samples, ChAT activity is normalized to total protein concentration, measured using Bradford protein assay (Bio-Rad) and is represented as nmol/min/mg of total protein.

$$Choline Depleted ( \frac{\frac{nmol}{min}}{m L} ) = \frac{[ ( [ Choline Blank ] ) - ( [ Plasma Sample ] ) ]}{Reaction Time ( mins ) * Sample Volume ( mL )} \times DF$$

For assays with α-NETA (Abcam), the diluted biological sample or recombinant protein was incubated with α-NETA (0-20 μM) at room temperature for 30 minutes prior to the colorimetric assay. For thermal denaturation, the recombinant protein or biological sample were heated in a thermal cycler at 70° C. or 95° C. respectively for 30 minutes at 1500×rpm.

Determination of Michaelis-Menten kinetic constants. To determine the Km constants for rChAT samples, a previously published Coenzyme A detection assay was used (12). Briefly, Coenzyme A standard was diluted in assay buffer. 100 μl of each standard and 40 μl of each sample was added to the 96-well plate in triplicate. 60 μl of Cocktail A was added to all sample wells. Immediately, 50 μl of Cocktail B was added to all sample and standard wells. The plate was immediately quantitated spectrophotometrically at 324 nm every 30 seconds for 30 minutes. Enzyme velocity was quantified as CoA formed (nmol/min/ml).

Animal Studies. All animal procedures were approved by the Institutional Animal Care and Use Committee and the Institutional Biosafety Committee of the Feinstein Institutes for Medical Research, Northwell Health, Manhasset, NY in accordance with NIH guidelines. Animals were kept at 25° C., 55-60% humidity, on a 12-h light-dark cycle with ad libitum access to food and water. Wild type, male, C57/B16 mice, 8-10 weeks of age, were purchased from Jackson Laboratory (Bar Harbor, ME) or Charles River (Wilmington, MA) and maintained in fully AAALAC accredited facilities at the Feinstein Institutes for Medical Research.

Endotoxemia. Lipopolysaccharide (LPS, Escherichia coli 0111: B4; Sigma) was sonicated for 30 min, vortexed and administered intraperitoneally (LD50 7 mg/kg). Animals were euthanized by CO₂ asphyxiation 90 min post-LPS administration and blood was collected by cardiac puncture using heparinized syringes. Blood samples were kept on ice and centrifuged for 10 minutes at 5000 g. Plasma TNF levels were quantified using a commercially available ELISA (Invitrogen). Plasma total protein concentration was determined using a Bradford Protein Assay (Bio-Rad). Sepsis scores were determined at respective timepoints up to 72 hours following endotoxin administration as described previously (19), where a maximum score of 28 per mouse denotes the greatest clinical sickness score.

Administration of PEG-ChAT and Anti-ChAT Neutralizing Antibody. Pegylated recombinant ChAT protein or a custom made monoclonal anti-ChAT antibody (GenScript) was administered intraperitoneally at a 1 mg/kg dose 30 minutes prior to LPS administration. Acute endotoxemia was induced with a dose of 0.3 mg/kg LPS. Animals were returned to their home cages and euthanized 90 minutes after LPS administration.

Cervical Vagus Nerve Stimulation. Aseptic technique was used for all surgical procedures. Vagus nerve isolation was carried out as previously described (20). Briefly, mice were anesthetized in a supine position with isoflurane (oxygen flow 2 L/min; isoflurane 1.5%). Cervical vagus nerve was isolated and placed on a custom-built bipolar cuff electrode (Microprobes) as described previously (20). Electrical pulses were delivered to the nerve using a constant impedance stimulator, MC Stim II using the following stimulation parameters: Four-minute duration, pulse width (16.1 ms) amplitude (250 μA) and frequency (30 Hz). The same surgical procedures were performed on sham operated mice but without electrical stimulation. After stimulation, the skin was stapled closed, and the animals were returned to their home cages for recovery. Two hours following stimulation, blood was collected by cardiac puncture using heparinized syringes.

DSS Colitis Model. DSS (DSS salt, colitis grade (MW: 36000-50000), MP Biomedicals, CA) was added to the drinking water in a final concentration of 3% (wt/vol) for 7 days. The water was changed to regular water on day 8 and maintained till the end of the experiment. Survival and the disease activity index (DAI) were evaluated daily in a blinded manner. On day 14, mice were euthanized under CO₂ asphyxiation and blood and tissue were collected. Colons were removed, cleaned, and sectioned longitudinally. The luminal contents were removed with cold phosphate buffered saline (PBS). Starting from the most distal end (rectum) the colon was rolled with luminal side facing upward, resulting in a “Swiss roll” with the distal end in the center and the proximal colon in the outer side of the roll. The Swiss roll was held together with a small pin and placed in 10% buffered formalin overnight at 4° C. The fixed tissue was then embedded in paraffin, sectioned at 10 μm and stained with hematoxylin and eosin. Histological analysis was carried out in a blinded manner by three independent researchers. The degree of colon inflammation was scored under the light microscope (Zeiss, Germany) using the following criteria: inflammation severity (0—none; 1—mild; 2—moderate; 3—severe), inflammation extent (0—none; 1—mucosal; 2—mucosal and submucosal; 3—transmural), Crypt damage (0—none; 1—basal, ⅓ damaged; 2—basal, ⅔/damaged; 3—crypt loss), damage distribution (0-0%; Jan. 1, 2025%; Feb. 26, 1950%; 3-51-75%; 4-76-100%). Each mouse tissue was scored over 6 different sites of the colon. Serum cytokine levels (IL-6, TNFα and MCP-1) were analyzed using mouse V-Plex Biomarker Group 1 array, and the assay was performed according to the manufacturer's instructions.

Human Plasma Samples. Blood samples were obtained from normal healthy controls and patients with sepsis or septic shock recruited to the Northwell Health System between 2018-2019 as described previously (21). All participants provided informed consent, and the study was approved by the institutional review board (IRB) of the Feinstein Institutes for Medical Research (Clinicaltrials.gov #NCT03389555). Patients were diagnosed with sepsis or septic shock by the Sepsis 3 criteria (22) and blood samples were obtained within 24 hours of the sepsis diagnosis (defined as “time 0”). Sample size was determined by availability, and no blinding or randomization was applied for these non-interventional observations. Sample cohort consisted of (n=11) septic patients with an age range of 62-94, and (n=7) control patients with an age range of 60-88 years old. ChAT activity was assessed in human plasma samples using the established assay.

Data analysis and statistics. Data were analyzed using Graphpad Prism 9.0 software using two-tailed unpaired Mann Whitney t-tests for comparisons between 2 groups or two-way ANOVA or mixed-effects model followed with Šídák's multiple comparisons test for comparisons of 3 or more groups. Michaelis Menten kinetic constants were determined using the Michaelis-Menten approximation to calculate Km and Vmax from a velocity vs. substrate relationship. Correlation coefficients were calculated using a nonparametric Spearman Correlation. For all analyses, P≤0.05 was considered statistically significant.

### Results

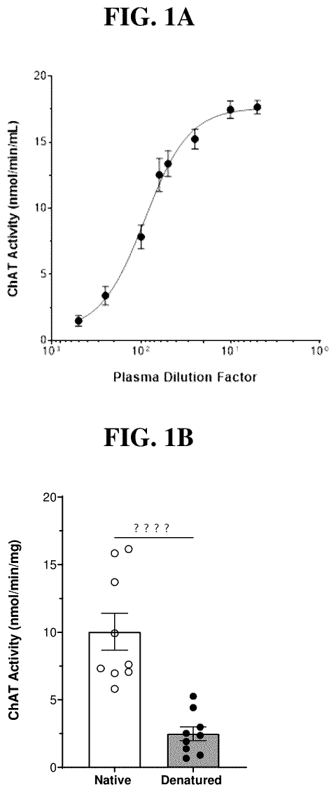

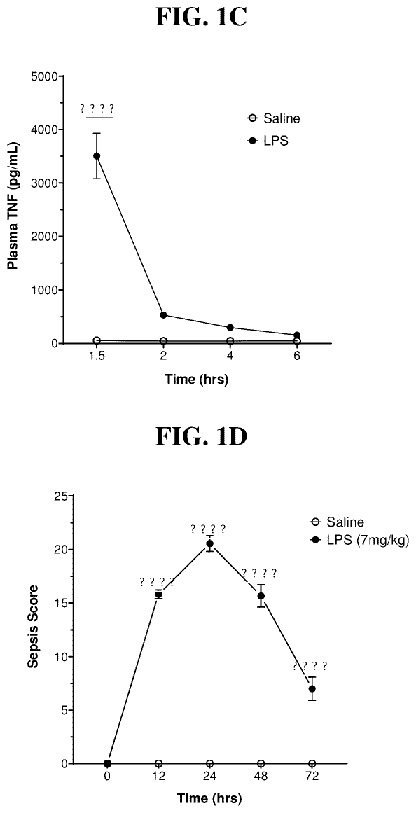

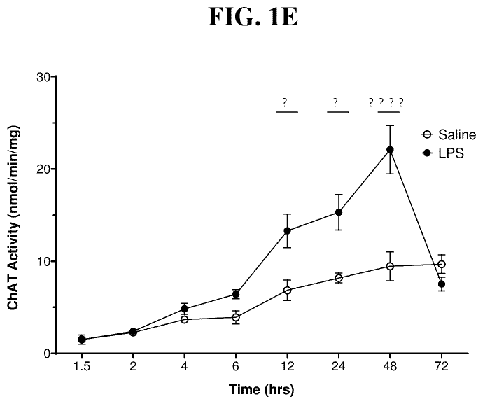

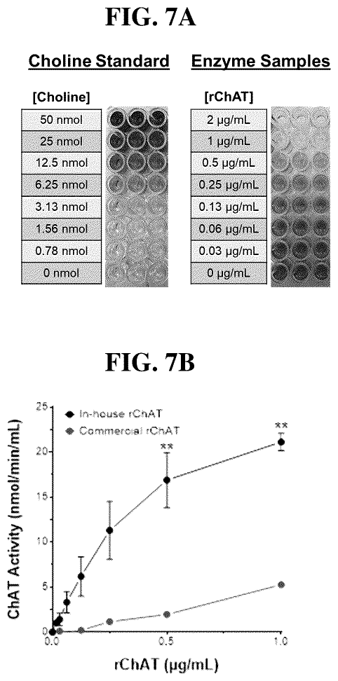

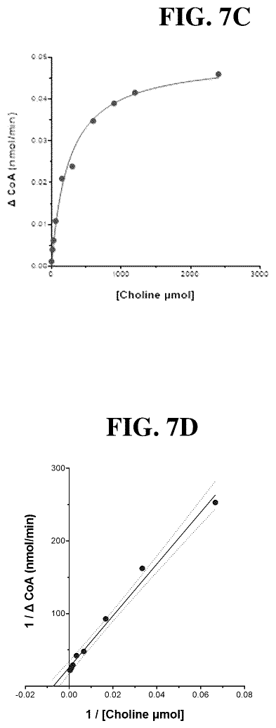

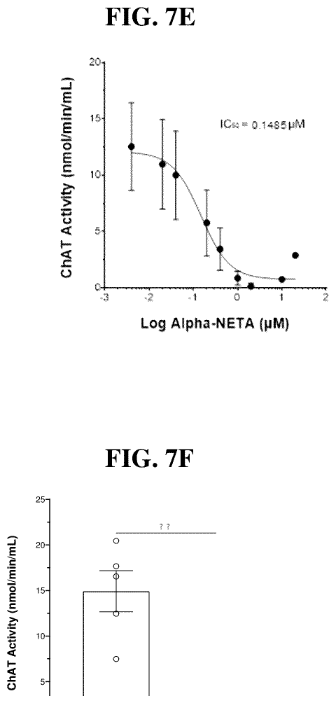

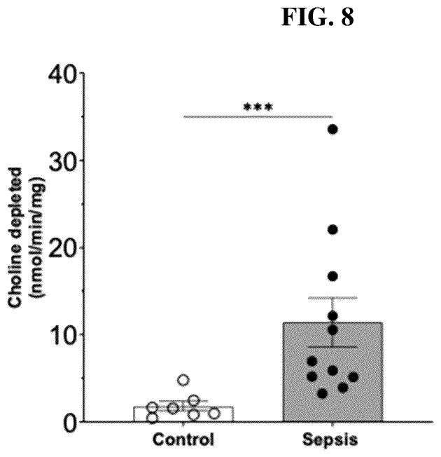

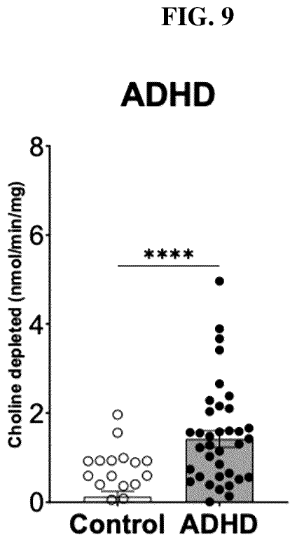

Active ChAT is present in normal mouse plasma. A novel enzyme assay was first established to measure the activity of ChAT in biological samples. Recombinant ChAT protein was expressed in Escherichia coli, purified to homogeneity (10), and used to establish the ChAT enzyme assay (FIG. 7). The relative activity of ChAT in plasma samples obtained from normal C57BL6 mice (8-12 weeks old) was then evaluated. A concentration-dependent increase in ChAT activity (FIG. 1A) is observed with a quasi-linear activity curve between dilutions of 1:200 and 1:50. Consistent with recombinant ChAT, plasma ChAT activity is significantly reduced after thermal denaturation at 95° C. (FIG. 1B).

Plasma ChAT activity increases following endotoxin challenge. To determine whether endogenous ChAT activity is altered in the setting of acute inflammation, a model of endotoxemia was established. Animals were exposed to a sublethal dose of endotoxin (LD50 dose, 7 mg/kg administered intraperitoneally, and plasma ChAT activity analyzed at varying time points over the following 72 hours. LPS administration significantly increased plasma TNF levels at 90 minutes (FIG. 1C) with a significant increase in sepsis score over time (FIG. 1D). In mice exposed to endotoxin, a significant increase in ChAT activity was observed beginning after 12 hours peaking by 48 hours before returning to baseline by 72 hours (FIG. 1E).

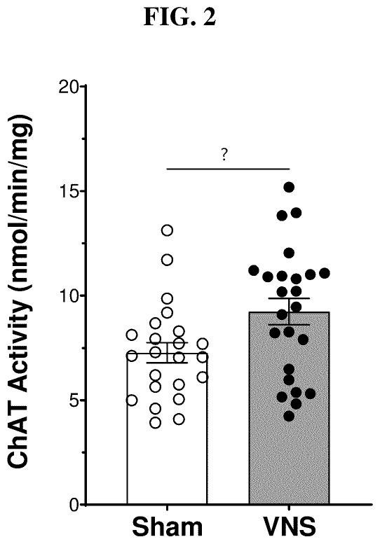

Circulating ChAT activity increases following vagus nerve stimulation. Activation of the cholinergic anti-inflammatory pathway by vagus nerve stimulation increases acetylcholine levels and requires ChAT-expressing T cells to attenuate TNF in endotoxemia (7). Further, activation of immune cells results in release of both acetylcholine and ChAT (5, 7, 13). Because vagus nerve stimulation increases acetylcholine levels (7), it was tested whether it may also result in increased circulating ChAT levels. To evaluate this possibility, plasma ChAT activity was measured in mice receiving sham versus electrical stimulation of the left cervical vagus nerve trunk (n=24 per group). ChAT activity levels are significantly elevated in plasma 2 hours after vagus nerve stimulation (FIG. 2). These data indicate that vagus nerve signaling enhances ChAT activity in circulation.

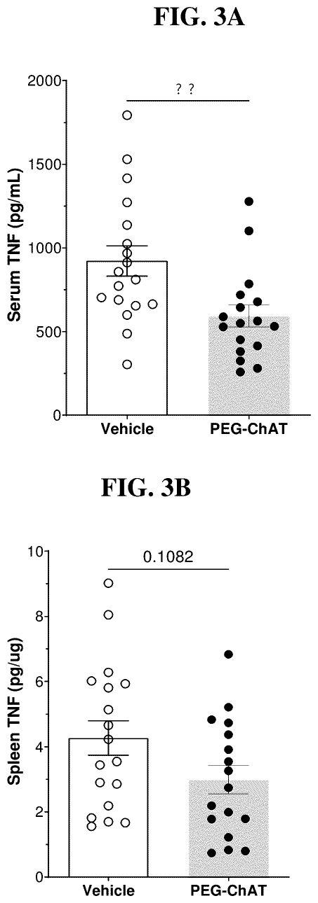

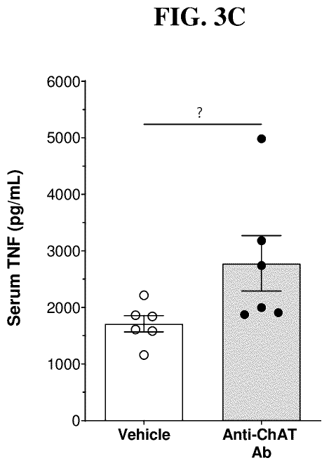

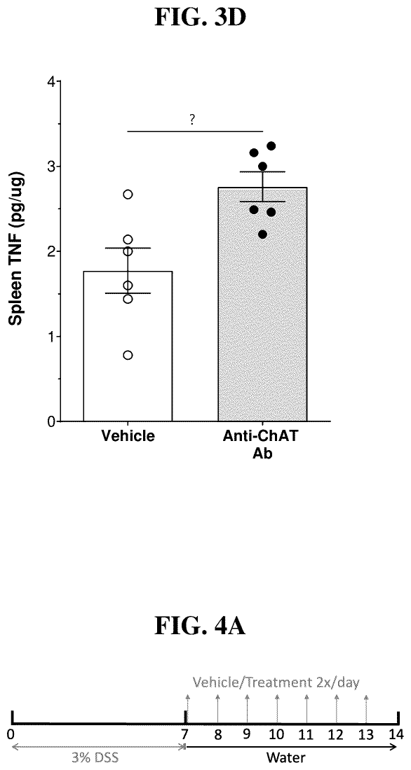

Circulating ChAT regulates inflammatory responses. To evaluate the role of circulating ChAT in regulating inflammatory responses, highly purified pegylated recombinant ChAT (PEG-ChAT, 1 mg/kg) was administered or vehicle was administered 30 minutes prior to LPS administration. Within 90 min, animals receiving vehicle exhibit increased levels of serum TNF, whereas administration of PEG-ChAT significantly attenuates serum TNF levels as compared to vehicle group (FIG. 3A). Administration of PEG-ChAT also induces a decrease in splenic TNF levels (FIG. 3B). To determine whether neutralization of circulating ChAT would exacerbate the disease, ChAT activity was neutralized using an antibody to ChAT. Passive immunization of un-anesthetized mice with a single dose of an anti-ChAT antibody (1 mg/kg) 30 min before a sublethal dose of LPS (0.3 mg/kg) significantly increases endotoxin-induced TNF levels in both the serum and spleen (FIG. 3C-D).

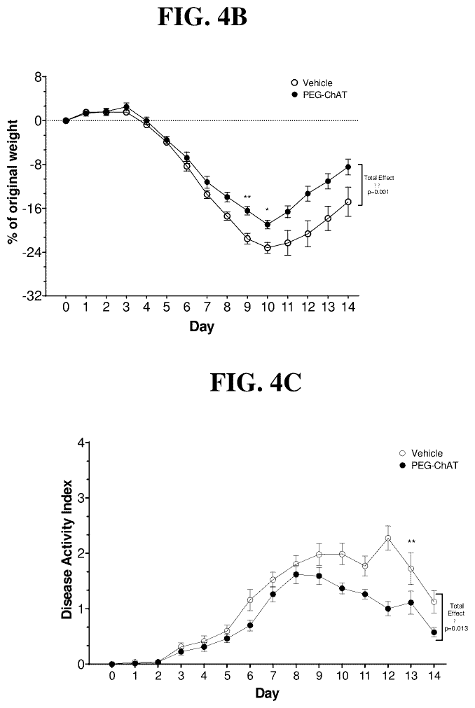

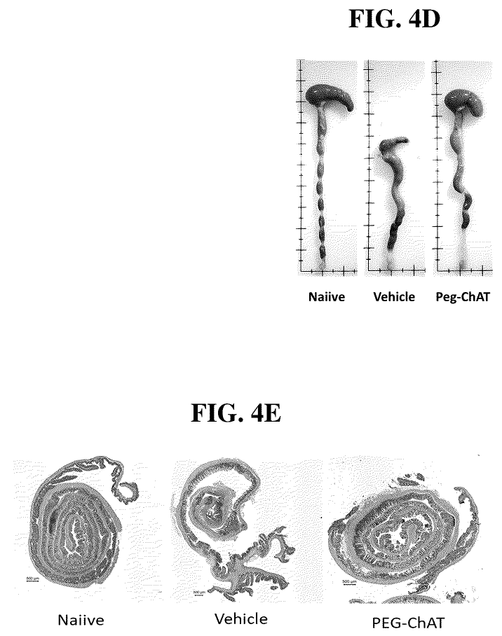

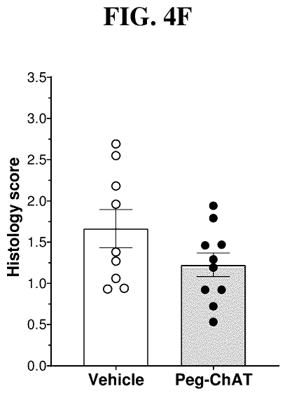

PEG-ChAT administration attenuates experimental colitis. Next, it was evaluated whether administration of PEG-ChAT ameliorates inflammation in active disease using the well-established DSS-colitis model. Mice were administered 3% DSS in the drinking water for 7 days, and body weight and disease activity monitored daily (FIG. 4A). DSS-exposed mice significantly lose weight from the 5th day after disease initiation (FIG. 4B). Intraperitoneal administration of PEG-ChAT (1 mg/kg) twice daily from day 7 to day 14 after disease induction significantly reduces severity of colitis. Animals receiving PEG-ChAT with colitis show a significant reduction in weight loss (FIG. 4B) and disease activity index (DAI) from day 9 as compared to vehicle-treated animals (FIG. 4C).

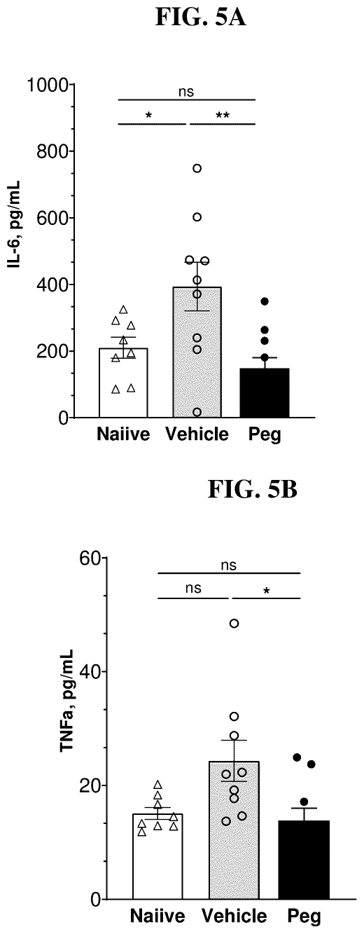

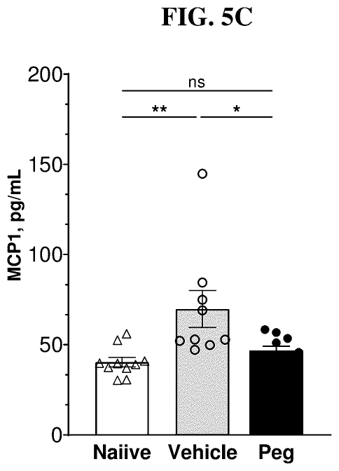

Shorter colon length and histological damage are considered as a hallmark of experimental colitis (23). DSS-exposed mice have significantly shortened colon (FIG. 4D). PEG-ChAT administration significantly improves colon length (FIG. 4D). To evaluate the effect of PEG-ChAT on histological damage, histopathological analysis was carried out in a blinded manner. Significantly greater damage to the epithelium and crypt, edema in mucosa and submucosa was observed in vehicle-treated mice, whereas PEG-ChAT administration attenuates histological damage (FIG. 4E-F). In addition, circulating levels of pro-inflammatory cytokines (IL-6, TNF and MCP-1) are significantly reduced in animals receiving PEG-ChAT as compared to vehicle controls (FIG. 5A-C).

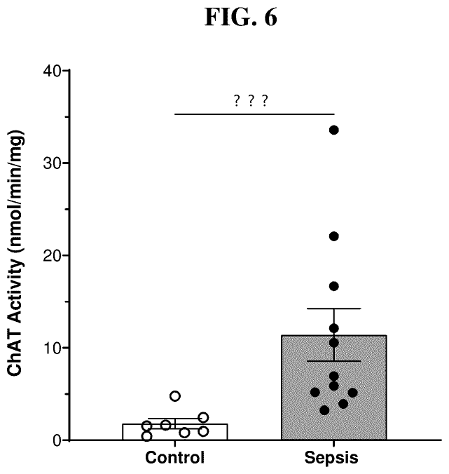

Circulating ChAT activity is increased in patients with sepsis. Animal models of human diseases, including the murine endotoxemia and colitis models used here, have inherent limitations (24-26). As an initial step in determining whether ChAT participates in the pathogenesis of human inflammatory diseases, 7 normal subjects and 9 critically ill septic patients were studied. Low levels of ChAT activity are detectable in the serum of normal subjects, but a significant increase is observed in critically ill patients with sepsis (FIG. 6), with a relatively higher level in a patient who succumbed as compared to patients with nonlethal infection.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

### REFERENCES

1. T. Fujii et al., Localization and synthesis of acetylcholine in human leukemic T cell lines. Journal of neuroscience research 44, 66-72 (1996).
2. T. Fujii et al., Constitutive expression of mRNA for the same choline acetyltransferase as that in the nervous system, an acetylcholine-synthesizing enzyme, in human leukemic T-cell lines. Neuroscience letters 259, 71-74 (1999).
3. T. Fujii, Y. Takada-Takatori, K. Kawashima, Basic and clinical aspects of non-neuronal acetylcholine: expression of an independent, non-neuronal cholinergic system in lymphocytes and its clinical significance in immunotherapy. J Pharmacol Sci 106, 186-192 (2008).
4. T. Fujii et al., Expression and Function of the Cholinergic System in Immune Cells. Front Immunol 8, 1085 (2017).
5. L. Tarnawski et al., Cholinergic regulation of vascular endothelial function by human ChAT+ T cells. Proceedings of the National Academy of Sciences 120, e2212476120 (2023).
6. M. A. Cox et al., Choline acetyltransferase-expressing T cells are required to control chronic viral infection. Science 363, 639-644 (2019).
7. M. Rosas-Ballina et al., Acetylcholine-synthesizing T cells relay neural signals in a vagus nerve circuit. Science 334, 98-101 (2011).
8. P. S. Olofsson et al., Blood pressure regulation by CD4 (+) lymphocytes expressing choline acetyltransferase. Nat Biotechnol 34, 1066-1071 (2016).
9. M. J. Schloss et al., B lymphocyte-derived acetylcholine limits steady-state and emergency hematopoiesis. Nature Immunology 23, 605-618 (2022).
10. A. Stiegler et al., Systemic administration of choline acetyltransferase decreases blood pressure in murine hypertension. Mol Med 27, 133 (2021).
11. G. L. Ellman, K. D. Courtney, V. Andres Jr, R. M. Featherstone, A new and rapid colorimetric determination of acetylcholinesterase activity. Biochemical pharmacology 7, 88-95 (1961).
12. L.-P. Chao, F. Wolfgram, Spectrophotometric assay for choline acetyltransferase. Analytical Biochemistry 46, 114-118 (1972).
13. S. Vijayaraghavan et al., Regulated Extracellular Choline Acetyltransferase Activity—The Plausible Missing Link of the Distant Action of Acetylcholine in the Cholinergic Anti-Inflammatory Pathway. PLOS One 8, e65936 (2013).
14. K. Kawashima, T. Fujii, The lymphocytic cholinergic system and its contribution to the regulation of immune activity. Life Sci 74, 675-696 (2003).
15. M. Mashimo, K. Kawashima, T. Fujii, Non-neuronal Cholinergic Muscarinic Acetylcholine Receptors in the Regulation of Immune Function. Biological and Pharmaceutical Bulletin 45, 675-683 (2022).
16. S. K. Kawashima, T. Fujii, Basic and clinical aspects of non-neuronal acetylcholine: overview of non-neuronal cholinergic systems and their biological. Journal of pharmacological sciences 106, 167-173 (2008).
17. K. Murray et al., Neuroanatomy of the spleen: Mapping the relationship between sympathetic neurons and lymphocytes. PLOS One 12, e0182416 (2017).
18. K. Fujimoto, M. Matsui, T. Fujii, K. Kawashima, Decreased acetylcholine content and choline acetyltransferase mRNA expression in circulating mononuclear leukocytes and lymphoid organs of the spontaneously hypertensive rat. Life sciences 69, 1629-1638 (2001).
19. B. Shrum et al., A robust scoring system to evaluate sepsis severity in an animal model. BMC research notes 7, 1-11 (2014).
20. H. A. Silverman et al., Standardization of methods to record Vagus nerve activity in mice. Bioelectronic Medicine 4, 1-13 (2018).
21. C. S. Zhu et al., Identification of procathepsin L (pCTS-L)-neutralizing monoclonal antibodies to treat potentially lethal sepsis. Science Advances 9, eadf4313 (2023).
22. M. Shankar-Hari et al., Developing a New Definition and Assessing New Clinical Criteria for Septic Shock: For the Third International Consensus Definitions for Sepsis and Septic Shock (Sepsis-3). JAMA 315, 775-787 (2016).
23. Y. H. Park et al., Adequate dextran sodium sulfate-induced colitis model in mice and effective outcome measurement method. Journal of cancer prevention 20, 260 (2015).
24. M. P. Fink, S. O. Heard, Laboratory models of sepsis and septic shock. Journal of Surgical Research 49, 186-196 (1990).
25. K. J. Tracey, E. Abraham, From mouse to man: or what have we learned about cytokine-based anti-inflammatory therapies? Shock 11, 224-225 (1999).
26. E. A. Deitch, Animal models of sepsis and shock: a review and lessons learned. Shock 10, 442-443 (1998).
27. A. Karami, T. Darreh-Shori, M. Schultzberg, M. Eriksdotter, CSF and plasma cholinergic markers in patients with cognitive impairment. Frontiers in Aging Neuroscience 13, 704583 (2021).
28. K. Kajiyama, T. Suzuki, K. Fujimoto, K. Kawashima, Determination of acetylcholine content and choline acetyltransferase activity in rabbit blood cells obtained from the buffy coat layer. Japanese Journal of Pharmacology 55, 194 (1991).
29. K. Kajiyama, K. Fujimoto, T. Suzuki, Y. Takada, K. Kawashima, Localization of acetylcholine and choline acetyltransferase activity in human mononuclear leucocytes. Japanese Journal of Pharmacology 58, 59 (1992).
30. I. Rinner, K. Schauenstein, Detection of choline-acetyltransferase activity in lymphocytes. Journal of neuroscience research 35, 188-191 (1993).
31. A. J. Knights et al., Acetylcholine-synthesizing macrophages in subcutaneous fat are regulated by β2-adrenergic signaling. The EMBO journal 40, e106061 (2021).
32. A. Hecker et al., Peripheral choline acetyltransferase is expressed by monocytes and upregulated during renal allograft rejection in rats. Journal of Molecular Neuroscience 30, 23 (2006).
33. C. J. Kirkpatrick, F. Bittinger, K. Nozadze, I. Wessler, Expression and function of the non-neuronal cholinergic system in endothelial cells. Life sciences 72, 2111-2116 (2003).
34. T.-G. Yi et al., A novel immunomodulatory mechanism dependent on acetylcholine secreted by human bone marrow-derived mesenchymal stem cells. International Journal of Stem Cells 12, 315-330 (2019).
35. Gabalski et al. Circulating extracellular choline acetyltransferase regulates inflammation. The Journal of Internal Medicine, 2024, 295:246-356.

What is claimed is:

1. A colorimetric assay for determining choline acetyl transferase (ChAT) activity in a biological sample comprising:
(i) obtaining or having obtained a biological sample to be assayed for the presence of ChAT activity;
(ii) performing a colorimetric assay for determining ChAT activity in the biological sample said colorimetric assay comprising (a) catalyzing the acetylation of the limiting substrate, choline, to form Ach in the presence of ChAT activity; (b) adding choline oxidase (“Choline Ox”) to oxidize the remaining choline to produce hydrogen peroxide; (c) adding horseradish peroxidase (“HRP”) to oxidizes the substrate 3,3′,5,5′-tetramethylbenzidine (“TMB”) relative to the quantity of hydrogen peroxide produced in the reaction; and (d) analyzing the reaction spectrophotometrically at 650 nm to determine the ChAT activity.

2. The colorimetric assay of claim 1, for determining ChAT activity in a biological sample, which comprises:
a. preparing at least one test sample by taking a biological sample and diluting said biological sample with Assay Buffer;
b. adding 40 μL of the at least one test sample to a reaction well in a multi-well reaction plate, which further comprises reaction wells that comprise 100 μL of at least one Reaction Standard;
c. adding 60 μL of Cocktail A to the at least one biological sample;
d. incubating the reaction plate at about 35-40° C. for about 15-20 minutes;
e. adding 50 μL of Cocktail B to all reaction wells;
f. incubating the reaction plate at room temperature for about 15-20 minutes;
g. adding 150 μL of 3,3′,5,5′-tetramethylbenzidine to all reaction wells; and
analyzing the reaction plate spectrophotometrically at 650 nm.

3. A method for screening a patient for a ChAT-biomarker associated disease, comprising: (i) performing the colorimetric assay of claim 1; and (ii) determining whether the patient has a ChAT-biomarker associated disease based upon the observed ChAT activity.

4. A method for screening a patient for a ChAT-biomarker associated disease, comprising: (i) performing the colorimetric assay of claim 2; and (ii) determining whether the patient has a ChAT-biomarker associated disease based upon the observed ChAT activity.

5. The method of claim 3, wherein the ChAT-biomarker associated disease is an inflammatory or cognitive disease.

6. The method of claim 5, wherein the ChAT-biomarker associated disease is selected from the group consisting of Alzheimer's disease, ADHD and sepsis.

7. The method according to claim 3, wherein the patient is a human.

8. The method according to claim 3, wherein the biological same is selected from the group consisting of blood, plasma, serum, tissues, isolated cells, cerebral spinal fluid, and cell supernatants.

9. A method for treating a patient suffering from a ChAT-biomarker associated disease comprising: (i) performing the colorimetric assay of claim 1; and (ii) administering an effective amount of a therapeutic agent to said patient based upon an observed alteration in ChAT activity.

10. A method for treating a patient suffering from a ChAT-biomarker associated disease comprising: (i) performing the colorimetric assay of claim 2; and (ii) administering an effective amount of a therapeutic agent to said patient based upon an observed alteration in ChAT activity.

11. The method of claim 9, wherein the ChAT-biomarker associated disease is an inflammatory or cognitive disease.

12. The method of claim 11, wherein the ChAT-biomarker associated disease is selected from the group consisting of Alzheimer's disease, ADHD and sepsis.

13. The method according to claim 9, wherein the patient is a human.

14. The method according to claim 9, wherein the biological same is selected from the group consisting of blood, plasma, serum, tissues, isolated cells, cerebral spinal fluid, and cell supernatants.

15. The method according to claim 9, wherein an effective amount of ChAT or ChAT-PEG is administered to the patient.

16. The method according to claim 9, wherein an effective amount of an anticholinergic agent is administered to the patient and the anticholinergic agent is donepezil, galantamine, or rivastigmine.

17. The method according to claim 16, wherein an effective amount of a ChAT antagonist is administered to the patient.

18. A method for screening a patient for a disease in which altered extracellular levels of ChAT are implicated comprising: (i) performing the colorimetric assay of claim 1; and (ii) determining whether the patient has said disease in which altered extracellular levels of ChAT are implicated.

19. A method for screening a patient for a disease in which altered extracellular levels of ChAT are implicated comprising: (i) performing the colorimetric assay of claim 2; and (ii) determining whether the patient has said disease in which altered extracellular levels of ChAT are implicated.

20. A method for treating a patient for a disease in which altered extracellular levels of ChAT are implicated comprising: (i) performing the colorimetric assay of claim 1; and (ii) determining whether the patient has said disease in which altered extracellular levels of ChAT are implicated and (iii) administering an effective amount of ChAT or a therapeutic agent recognized to treat said disease to said patient based upon the choline acetyl transferase activity.

21. A method for treating a patient for a disease in which altered extracellular levels of ChAT are implicated comprising: (i) performing the colorimetric assay of claim 2; and (ii) determining whether the patient has said disease in which altered extracellular levels of ChAT are implicated and (iii) administering an effective amount of ChAT or a therapeutic agent recognized to treat said disease to said patient based upon the choline acetyl transferase activity.

22. A kit for determine the ChAT activity in a biological sample, which comprises Cocktail A, Cocktail B, and Assay Buffer.
